(19) United States
(12) Patent Application Publication
(10) Pub. No.: US 2024/0337599 A1
(43) Pub. Date: Oct. 10, 2024

(54) SYSTEMS AND METHODS FOR PREDICTING SOIL PROPERTIES

(71) Applicant: TERRAMERA, INC., Vancouver (CA)

(72) Inventors: Austin Caulfield ROTHWELL, Vancouver (CA); Travis Godwin GOOD, Vancouver (CA); Hoda AGHAEI, Vancouver (CA); Maryamsadat RASOULIDANESH, Vancouver (CA); Sadegh SHOKATIAN, Vancouver (CA); Vinod PARMAR, Vancouver (CA); Conor Andrew SEXTON, Vancouver (CA)

(21) Appl. No.: 18/744,377

(22) Filed: Jun. 14, 2024

Related U.S. Application Data
(63) Continuation of application No. PCT/CA2022/051829, filed on Dec. 15, 2022.
(60) Provisional application No. 63/291,151, filed on Dec. 17, 2021.

(51) Int. Cl. G01N 21/65; G06N 3/0475

(52) U.S. Cl. CPC G01N 21/65; G01N 33/245; G06N 3/0475

(57) ABSTRACT

Machine learning models for generating predictions of soil properties based on Raman spectral measurements are provided. The models can be trained on synthetic training data with associated synthetic Raman spectral measurements and ground truth training data with associated ground truth Raman spectral measurements. Systems and methods for training such machine learning models are also provided. Predictions may be facilitated by generating synthetic spectral measurements by physical simulation for training data, using spectral measurements at varying degrees of resolution (e.g. using satellite measurements bolstered by measurements from aerial and/or proximal sensors), and/or modeling uncertainty in the predictions. Soil properties May comprise sequestered soil carbon, nitrogen, phosphorous, potassium, and other constituents. Synthetic and ground-truth modes of training may differ in the number and type of input provided.

### CROSS-REFERENCE TO RELATED APPLICATIONS

This application is a continuation of Patent Cooperation Treaty (PCT) application No. PCT/CA2022/051829 having an international filing date of 15 Dec. 2022, which in turn claims priority to, and the benefit of, U.S. provisional patent application No. 63/291,151 filed 17 Dec. 2021 entitled Systems and Methods for Predicting Soil Properties. All of the applications in this paragraph are hereby incorporated herein by reference herein for all purposes.

### TECHNICAL FIELD

The present disclosure relates generally to a method of predicting properties of soil, and particularly to a method of predicting soil properties spectrographically based on machine learning techniques.

### BACKGROUND

Quantifying soil properties is a challenging technical problem with significance to certain fields. For instance, quantifying soil nutrient content, such as nitrogen, phosphorous, and potassium soil contents, has significance in agriculture, horticulture, silviculture, and other fields. As another example, quantifying soil carbon content has significance in global efforts against climate change. Soils can store constituents, including carbon, nitrogen, phosphorous, and potassium, in various forms and such storage can be promoted by human interventions, such as certain agricultural or other land use-specific practices. In the context of soil carbon, such practices are sometimes incentivized through the use of carbon credit markets. In the context of various constituents, including plant nutrients, such practices are sometimes desirable for reasons of promoting plant health, crop yields, and the like. Practitioners of such practices can benefit from the ability to quantify soil properties with high accuracy, high scale (e.g. across arcas of tens to millions of square kilometers), and at low cost.

Traditional approaches for soil property quantification typically involves sending a sampling team to the area of interest, manually collecting soil samples, preparing the samples (e.g. via drying, sieving, and freezing), and shipping the prepared samples to a laboratory for characterization, e.g. via chemical analysis such as by dry combustion or acid treatment. Such approaches tend to be highly accurate, but tend to be laborious, costly, and slow-for instance, quantifying soil carbon content of one field can cost thousands of dollars and take months for results to come back, rendering it impractical for use at high scale.

Others have applied spectral approaches by measuring reflectance spectra of surface-level soils in the visible, near-IR, and/or short-wave IR spectral ranges and interpreting the measured spectra via a model to estimate soil properties. For example, Soriano-Disla et al. disclose soil spectroscopy methods for predicting certain soil properties involving obtaining diffuse reflectance infrared spectroscopy measurements of soils using handheld sensors in the visible-near infrared and mid-infrared spectral ranges and generating from those measurements predictions of soil properties (see Soriano-Disla et al., The Performance of Visible, Near-, and Mid-Infrared Reflectance Spectroscopy for Prediction of Soil Physical, Chemical, and Biological Properties. Appl. Spectrosc. Rev. 2014, 49, 139-186). Such methods can achieve reasonable accuracy and moderate scalability, but tend to involve calibrating based on large calibration datasets of laboratory-tested samples from each region in which spectral measurements are to be applied. Moreover, such methods can have reduced accuracy for certain soil properties, such as for soil organic matter.

Others have applied spectral approaches in remote sensing contexts, such as predicting soil carbon content based on satellite imagery. Such methods tend to be noisy and relatively lower-accuracy than the above-described techniques. For instance, Meng et al. disclose a remote sensing approach for predicting soil organic matter content at a regional scale using satellite reflectance hyperspectral data (see Meng et al., Soil Organic Matter Prediction Model with Satellite Hyperspectral Image Based on Optimized Denoising Method, Remote Sens. 2021, 13, 2273. https://doi.org/10.3390/rs13122273).

There is a general desire for soil property prediction systems and methods for generating predictions of soil properties with improved accuracy, at improved scale, and/or at reduced cost.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

One aspect of the invention provides systems and methods for predicting soil properties. The systems comprise one or more processors and a memory storing instructions which cause the one or more processors to perform operations comprising the methods. The methods are performed by a processor and comprise: obtaining an input Raman spectral measurement of soil; generating a first prediction of a soil property for the soil by a prediction model (referred to herein as a “prediction model”) configured to generate predictions of the soil property based on input Raman spectral measurement and having parameters trained over soil property training data and associated soil property training Raman spectral measurements.

In some embodiments, the Raman training data and associated Raman training spectral measurements comprise: synthetic sequestered soil property training data and associated synthetic training Raman spectral measurements; and ground truth sequestered soil property training data and associated ground truth training Raman spectral measurements. In some embodiments, the synthetic soil property training data comprises a measure of the soil property in a plurality of simulated soil samples, and the ground truth soil property training data comprises a measure of the soil property in a plurality of ground-truth soil samples. In some embodiments, the synthetic training Raman spectral measurements comprises a simulated Raman spectral measurement generated by a physical soil simulation. In some embodiments, at least one simulated Raman spectral measurement for a physical soil simulation comprising first and second soil components is based on a sum of a first simulated Raman spectral measurement for the first soil component and a second simulated Raman spectral measurement for the second soil component.

In some embodiments, the simulated Raman spectral measurement is based on a simulation of at least one of: a vibrational frequency and a vibrational intensity of molecules represented in the physical soil simulation.

In some embodiments, the synthetic training Raman spectral measurement comprises a predicted Raman spectral measurement generated by a generative machine learning model trained over soil properties and configured to predict Raman spectral measurements based on soil properties.

In some embodiments, the ground truth soil property training data corresponds to a plurality of ground truth training locations in an area of interest; the prediction model is configured to generate predictions of the soil property based on the input Raman spectral measurement and a location identifier associated with the input Raman spectral measurement, the prediction model having parameters trained over a plurality of ground-truth location identifiers associated with the ground truth training Raman spectral measurements; and generating the first prediction of the soil property for the soil comprises generating the first prediction of the soil property for the location based on a location identifier for the soil associated with the input Raman spectral measurement. In some embodiments, the first portion of the prediction model has parameters trained over synthetic soil property training data and associated synthetic training Raman spectral measurements; and the second portion of the prediction model has parameters trained over synthetic soil property training data and associated synthetic training Raman spectral measurements and ground-truth soil property training data and associated ground-truth training Raman spectral measurements.

In some embodiments, the input Raman spectral measurement comprises a first Raman spectral signature corresponding to mineral-associated organic material and a second Raman spectral signature corresponding to particulate organic material, the method further comprising distinguishing at least a portion of the first spectral signature from the second spectral signature.

In some embodiments, the first prediction of the soil property comprises a predicted measure at least one of: soil carbon content, nitrogen content, potassium content, sulfur content, hydrogen content, nitrate content, ammonia content, phosphate content, aluminum content, iron content, phosphorus content, calcium content, magnesium content, sodium content, phospholipid fatty acid content, pH, carbon-nitrogen ratio, water content, water-soluble organic carbon content, and water-soluble organic nitrogen content. In some embodiments, the first prediction of the soil property comprises a predicted measure of sequestered soil carbon content for the soil. In some embodiments, the predicted measure of sequestered soil carbon content comprises a measure of mineral associated organic matter content.

In some embodiments, the method comprises generating a second prediction of non-sequestered soil carbon content for the soil. In some embodiments, the method further comprises generating a third prediction of total soil carbon content for the soil, the third prediction of total soil carbon content being based on a sum of soil carbon content of the first and second predictions.

In some embodiments, the input Raman spectral measurement comprises a high-spatial-resolution spectral measurement and a low-spatial-resolution spectral measurement, at least one of the high-spatial-resolution spectral measurement and low-spatial-resolution spectral measurement comprising a Raman spectral measurement. In some embodiments, the low-spatial-resolution spectral measurement comprises at least one of: satellite images and aerial images of an area of interest comprising a location of the soil.

In some embodiments, the high-resolution spectral measurement comprises an at-depth Raman spectral measurement of the soil. In some embodiments, the at-depth Raman spectral measurement comprises a spectral measurement of a soil sample obtained at a depth of at least one of: about 0.01 m to 1 m, 0.1 m to 1 m, 0.25 m to 1 m, and 0.1 m to 10 m.

In some embodiments, the first prediction comprises a measure of uncertainty. In some embodiments, wherein the machine learning model comprises a Bayesian ensemble neural network configured to provide the measure of uncertainty for each prediction.

In some embodiments, generating the first prediction of comprises generating an intermediate representation by a first portion of the prediction model, combining the intermediate representation with one or more additional inputs to form a combined input, and generating the first prediction by a second portion of the prediction model based on the combined input.

Aspects of the present disclosure provide systems and methods for training a machine learning model to predict soil properties. The systems comprise one or more processors and a memory storing instructions which cause the one or more processors to perform operations comprising the methods. The method is performed by a processor and comprises: obtaining training Raman spectral measurements based on soil samples; generating a plurality of predictions of a soil property by a machine learning model (referred to herein as a “prediction model”) based on the training Raman spectral measurements; determining a value of an objective function based on the plurality of predictions of soil properties and soil property training data associated with the training Raman spectral measurements; and modifying parameters of the prediction model based on the value of the objective function.

In some embodiments, the training Raman spectral measurements comprise synthetic training Raman spectral measurements based on a physical soil simulation of a simulated soil sample. In some embodiments, the method comprises generating the synthetic Raman spectral measurements by simulating at least one of: a vibrational frequency and a vibrational intensity of molecules represented in the simulated soil sample. In some embodiments, generating the synthetic training Raman spectral measurements comprises obtaining one or more soil properties and generating the physical soil simulation comprising the simulated Raman spectral measurement based on the one or more soil properties.

In some embodiments, at least one simulated Raman spectral measurement for the simulated soil sample comprising first and second soil components is based on a sum of a first simulated Raman spectral measurement for the first soil component and a second simulated Raman spectral measurement for the second soil component.

In some embodiments, the synthetic soil property training data comprises a predicted Raman spectral measurement generated by a generative machine learning model trained over soil properties and configured to predict Raman spectral measurements based on soil properties. In some embodiments, obtaining synthetic training Raman spectral measurements based on simulated soil samples comprises obtaining one or more soil properties and generating the predicted Raman spectral measurement by the generative machine learning model based on the one or more soil properties. In some embodiments, the generative machine learning model comprises a generative adversarial network.

In some embodiments, the training Raman spectral measurements comprise ground truth training spectral measurements based on soil samples from a plurality of sampling locations in an area of interest. In some embodiments, the soil property training data comprises a measure at least one of: soil carbon content, nitrogen content, potassium content, sulfur content, hydrogen content, nitrate content, ammonia content, phosphate content, aluminum content, iron content, phosphorus content, calcium content, magnesium content, sodium content, phospholipid fatty acid content, pH, carbon-nitrogen ratio, water content, water-soluble organic carbon content, and water-soluble organic nitrogen content. In some embodiments, the soil property training data comprises a measure of sequestered soil carbon content. In some embodiments, the soil property training data comprises a measure of mineral associated organic matter content.

In some embodiments, at least one training Raman spectral measurement comprises a high-spatial-resolution spectral measurement and a low-spatial-resolution spectral measurement. In some embodiments, the low-spatial-resolution spectral measurement comprises at least one of: a satellite image and an aerial image of at least a portion of the area of interest. In some embodiments, the high-resolution spectral measurement comprises an at-depth spectral measurement of soil at one of the sampling locations. In some embodiments, the at-depth spectral measurement comprises a spectral measurement of a soil sample obtained at a depth of at least one of: about 0.01 m to 1 m, 0.1 m to 1 m, 0.25 m to 1 m, and 0.1 m to 10 m.

In some embodiments, the training Raman spectral measurement comprises a first spectral signature corresponding to mineral-associated organic material and a second spectral signature corresponding to particulate organic material, the method further comprising distinguishing at least a portion of the first spectral signature from the second spectral signature.

In some embodiments, obtaining training Raman spectral measurements based on soil samples comprises obtaining synthetic training Raman spectral measurements based on simulated soil samples and obtaining ground truth training spectral measurements based on soil samples from the plurality of sampling locations in the area of interest; generating the plurality of predictions comprises generating a first plurality of predictions of the soil property by the prediction model based on the synthetic training Raman spectral measurements and generating a second plurality of predictions of the soil property for the plurality of sampling locations by the prediction model based on the ground truth training Raman spectral measurements; determining the value of an objective function comprises determining the first value of a first objective function based on the first plurality of predictions of soil properties and synthetic soil property training data associated with the synthetic training Raman spectral measurements and determining a second value of at least one of the first objective function and a second objective function based on the second plurality of predictions of the soil property and ground truth soil property training data associated with the ground truth training Raman spectral measurements; and modifying parameters of the prediction model comprises modifying parameters of the prediction model based on the first value and modifying parameters of the prediction model based on the second value.

In some embodiments, generating the first plurality of predictions of the soil properties by the prediction model based on the synthetic training Raman spectral measurements comprises, for at least one of the first plurality of predictions: generating a first intermediate representation by a first component of the prediction model based on at least one of the synthetic training Raman spectral measurements; and generating the at least one of the first plurality of predictions based on the intermediate representation; and generating the second plurality of predictions of the soil property by the prediction model based on the ground-truth training Raman spectral measurements comprises, for at least one of the second plurality of predictions: generating a second intermediate representation by the first component of the machine learning model based on at least one of the ground-truth training Raman spectral measurements; combining the intermediate representation with one or more additional inputs to form a combined input; and generating the at least one of the second plurality of predictions by a second component of the prediction model based on the combined input.

In some embodiments, the one or more additional inputs comprises at least one of: a location identifier, a terrain gradient, an aspect, a convergence index, a hill shade, a multi-resolution right top flatness, a multi-resolution valley bottom flatness, a standardized water level index, a valley depth, an elevation, a different from the mean elevation, a deviation from the mean elevation, a relative terrain index, a profile curvature, and a planform curvature associated with the at least one of the ground-truth training Raman spectral measurements.

In some embodiments, combining the intermediate representation with one or more additional inputs to form the combined input comprises concatenating the intermediate representation with at least one of the one or more additional inputs.

In some embodiments, at least one of the plurality of predictions comprises a measure of uncertainty. In some embodiments, the machine learning model comprises a Bayesian ensemble neural network configured to provide the measure of uncertainty for each prediction.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1A schematically shows an example system for training an example machine learning model for predicting soil properties in a first training mode based on simulated soil samples according to the present disclosure.

FIG. 1B schematically shows an example system for training the example machine learning model of FIG. 1A in a second training mode based on ground-truth soil samples.

FIG. 2 schematically shows an example system for generating predictions of soil properties with the example machine learning model of FIG. 1A.

FIG. 3 is a flowchart of an example method for training an example machine learning model for predicting soil properties, such as by the systems of FIG. 1A and FIG. 1B.

FIG. 4 is a flowchart of an example method for generating predictions of soil properties with an example machine learning model for predicting soil properties, such as by the system of FIG. 2.

FIG. 5 shows a first exemplary operating environment that includes at least one computing system for performing methods described herein, such as the methods of FIGS. 3 and 4.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

Soil properties may comprise measures of soil fractions, such as fractions of carbon (including soil organic carbon, soil inorganic carbon, sequestered soil carbon, etc.), nitrogen, potassium, sulfur, hydrogen, nitrate, ammonia, phosphate, aluminum, iron, phosphorus, calcium, magnesium, and/or sodium content; phospholipid fatty acid content; pH; carbon-nitrogen ratio; Haney test inputs and outputs (which may include certain of the foregoing and/or other features such as water-soluble organic carbon and/or organic nitrogen); and/or other soil properties

Soil carbon can be stored in soils relatively transiently, e.g. as particulate organic matter (POM), or relatively more durably, e.g. as mineral-associated organic matter (MAOM) or inorganic carbon. More durably stored forms of soil carbon tend to be preferred in contexts focused on addressing climate change. Such forms of soil carbon are referred to herein as “sequestered” soil carbon.

Systems and methods disclosed herein provide machine learning models which generate predictions of soil properties and are trained on training data comprising Raman spectral measurements. The machine learning models may be trained over synthetic training data with associated synthetic Raman spectral measurements and/or ground truth training data and with associated Raman ground truth spectral measurements. Systems and methods for training such machine learning models are also provided.

Using Raman spectral measurements can, in suitable circumstances, facilitate prediction of soil properties. Raman spectral measurements tend to be less sensitive to variations in angle of measurement, less sensitive to variations in soil water content, and more sensitive to differences in carbon fractions (e.g. MAOM vs. POM carbon) than at least some reflectance spectral measurements and in suitable circumstances may assist in improving accuracy of predictions of soil properties. In some embodiments, prediction of soil properties is facilitated by generating synthetic Raman spectral measurements by physical simulation of training data, which in suitable circumstances may assist with reducing the quantity of ground-truth samples required for training and/or with improving accuracy of predictions by trained models. In some embodiments, prediction of soil properties is facilitated by using Raman spectral measurements at varying degrees of resolution (e.g. using satellite measurements bolstered by measurements from aerial and/or proximal sensors), and/or modeling uncertainty in the predictions.

### Systems for Training a Sequestered Soil Properties Prediction Model

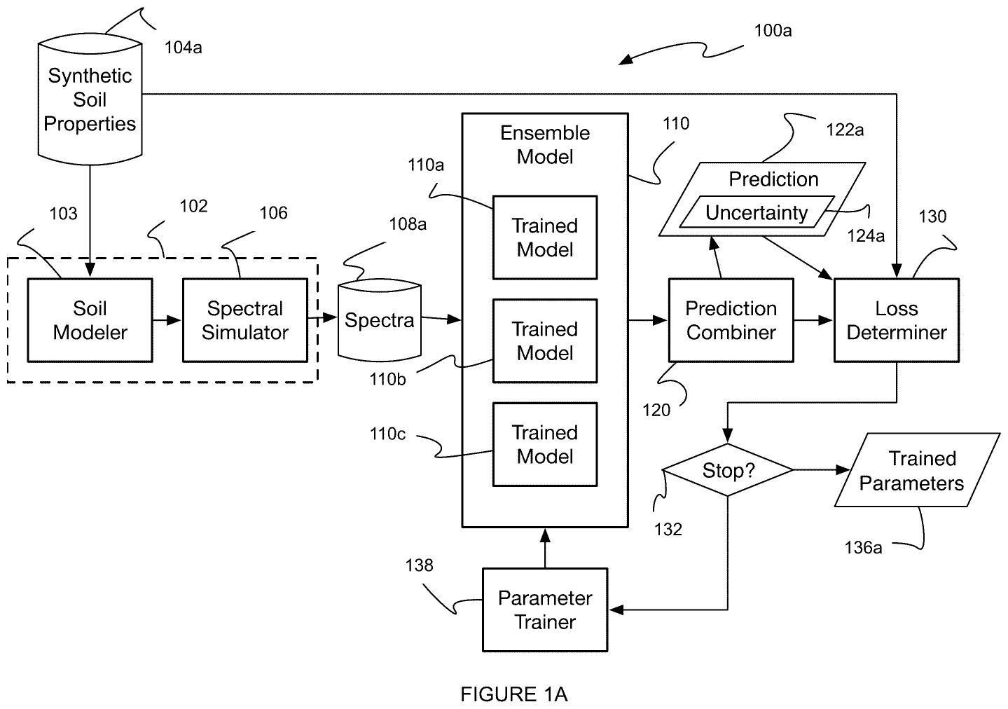

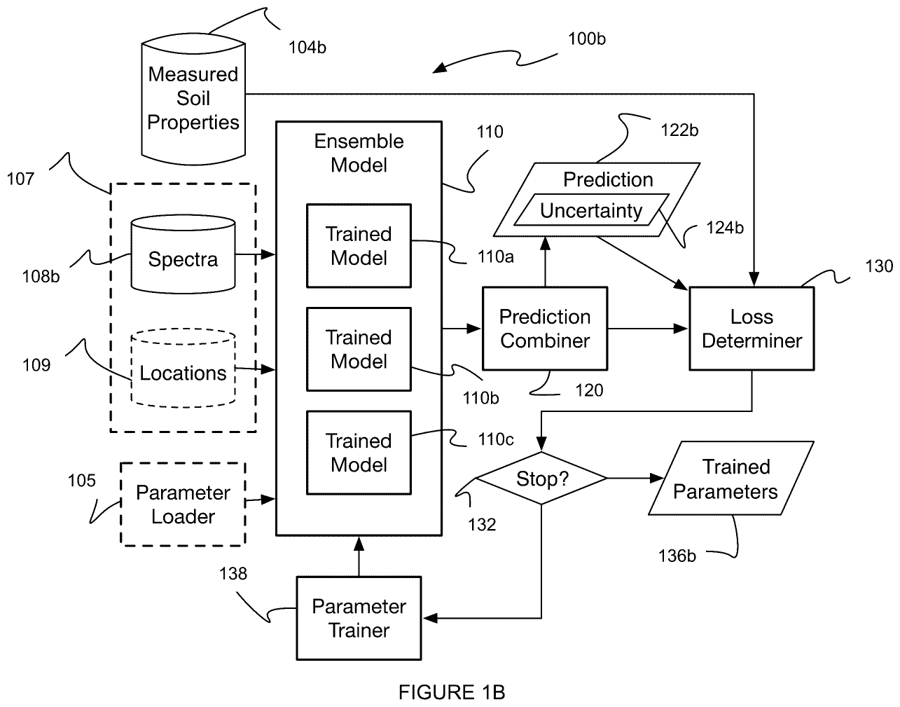

FIG. 1A schematically shows an example system 100a for training an example machine learning model 110 for predicting soil properties in a first training model based on simulated soil samples according to the present disclosure.

In some embodiments, system 100a comprises a synthetic soil spectral generator 102 for generating synthetic spectra of simulated soil samples. Synthetic soil spectral generator 102 receives synthetic soil properties 104a and generates synthetic spectral measurements 108a based on synthetic soil properties 104a. Synthetic soil spectra 108a may comprise simulated Raman spectral measurements. Synthetic soil properties 104a may comprise a representation of carbon content, e.g. specifying 5% carbon content for a simulated soil sample, specifying 3% carbon content stored as mineral-associated organic matter and 2% carbon content stored as particulate organic matter, and/or as other carbon fractions of a simulated soil sample. Synthetic soil properties 104a may additionally, or alternatively, comprise representations of other soil properties, such as fractions of nitrogen, potassium, sulfur, hydrogen, nitrate, ammonia, phosphate, aluminum, iron, phosphorus, calcium, magnesium, and/or sodium content; phospholipid fatty acid content; pH; carbon-nitrogen ratio; Haney test inputs and outputs (which may include certain of the foregoing and/or other features such as water-soluble organic carbon and/or organic nitrogen); and/or other soil properties.

Synthetic soil spectral generator 102 may comprise a soil modeler 103 and a spectral simulator 106 (e.g. as shown in the example embodiment of FIG. 1A). Soil modeler 103 receives synthetic soil properties 104a for a simulated soil sample, such as a representation of soil properties as described elsewhere herein, and generates a soil model for the simulated soil sample based on synthetic soil properties 104a. The soil model may comprise, for example, a representation of atoms, functional groups, molecular building blocks, molecules, and/or groups of molecules in the simulated soil sample. For instance, soil modeler 103 may comprise molecular building blocks as provided by Vienna Soil Organic Modeler 2 (e.g. as described by Escalona et al., Vienna Soil Organic Matter Modeler 2 (VSOMM2), J. Mol. Graph. Model. (2021) 103, 107817, doi: 10.1016/j.jmgm.2020.107817).

Spectral simulator 106 generates one or more synthetic spectral measurements 108a for the simulated soil sample based on the soil model. In some embodiments, simulated Raman spectral measurements 108a are generated by physical simulation of soil, e.g. by simulating molecular dynamical properties and/or quantum mechanical properties of the simulated soil sample, normal mode analysis (e.g. such as complex-based normal mode analysis). In some embodiments, spectral simulator 106 simulates Raman spectral measurements for the simulated soil sample by simulating vibrational frequency and vibrational intensity of molecules represented in the soil model based on polarizability of the molecules. In some embodiments, spectral simulator 106 additionally or alternatively simulates IR spectra for the simulated soil sample by simulating vibrational frequency and vibrational intensity of molecules represented in the soil model based on changes in dipole moments of the molecules. For instance, molecular dynamics properties and/or quantum mechanical properties may be simulated via the GROMACS and/or Gaussian™ (from Gaussian, Inc.) computational chemistry software packages. In some embodiments, physical soil simulation comprises simulating interactions between molecules based on normal mode analysis, such as complex-based normal mode analysis, based on elastic network models. For instance, molecular interactions may be modeled based on an anisotropic network model. In some embodiments, spectral simulator 106 simulates more than one type of spectra; for example, spectral simulator 106 may simulate two or more of: Rayleigh, Raman, IR, reflectance, and/or other types of spectra.

In some embodiments, synthetic soil spectral generator 102 generates models of soil components for a simulated soil sample, generates synthetic spectra for at least some of the soil components, and synthesizes a synthetic spectral measurement 108a by combining the synthetic spectra for the soil components. For example, soil modeler 103 may receive a set of soil properties 104a (e.g. indicating 5% soil carbon content) and generate a soil model comprising several soil components (e.g. molecules). Spectral simulator 106 may generate (and/or receive from a cache, e.g. if it has previously generated) synthetic spectral measurements for some or all of the soil components, and may combine those spectral measurements (e.g. by summation) to form a synthetic spectral measurement 108a, such as a synthetic Raman spectral measurement, for the simulated soil sample. Although some interactions between molecules may be lost in such a modeling approach, synthetic spectral measurements 108a may be more efficiently and quickly produced. At least in the contexts of Raman and IR spectra (though not limited to those contexts), loss in accuracy of the synthetic soil spectral measurements 108a under such an approach may be relatively small, and perhaps even negligible in suitable circumstances. Synthetic soil spectral measurements 108a may comprise one or more of Raman, IR, reflectance, and/or another type of spectral measurement.

In some embodiments, synthetic soil spectral generator 102 comprises a machine learning model. The machine learning model may comprise a generative machine learning model, such as a generative adversarial network (GAN), an invertible network, and/or any other suitable model. The machine learning model may be trained over ground-truth and/or simulated soil sample data to generate synthetic spectral measurements 108a based on one or more synthetic soil properties 104a. In at least some embodiments, soil properties 104a comprise a measure of soil properties in a form corresponding to a measure of soil properties provided by prediction 122a to facilitate training of machine learning model 110. Training machine learning model 122a may be performed separately from training the machine learning model of soil spectral generator 102 and may comprise comparing predicted soil properties of prediction 122a for a given simulated soil sample to the soil properties of soil properties 104a for that simulated soil sample and determining a loss (e.g. as described elsewhere herein with reference to loss determiner 130) based on that comparison.

Machine learning model 110 receives a synthetic spectral measurement 108a, such as a synthetic Raman spectral measurement, and generates a prediction 122a of soil properties for the simulated soil sample associated with the received synthetic spectral measurement 108a. Prediction 122a may comprise a prediction of any suitable soil property, including (but not limited to) measures of carbon (e.g. sequestered carbon), nitrogen, potassium, sulfur, hydrogen, nitrate, ammonia, phosphate, aluminum, iron, phosphorus, calcium, magnesium, and/or sodium content; phospholipid fatty acid content; pH; carbon-nitrogen ratio; Haney test inputs and outputs (which may include certain of the foregoing and/or other features such as water-soluble organic carbon and/or organic nitrogen); and/or other soil properties.

In some embodiments, including the depicted embodiment, the machine learning model comprises an ensemble model comprising a plurality of sub-models 110a, 110b, 110c, etc. (An ensemble machine learning model 110 may comprise any number of sub-models.) In some embodiments, predictions of sub-models 110a, 110b, 110c are combined by prediction combiner 120 to generate prediction 122a, e.g. by determining an average (e.g. a weighted average) and/or by any other suitable combination technique in the art.

In at least some embodiments, machine learning model 110 is configured to provide a measure of uncertainty 124a for prediction 122a. For instance, machine learning model 110 may comprise a Bayesian ensemble neural network configured to provide a measure of uncertainty 124a for prediction 122a based on predictions by sub-models 110a, 110b, 110c (e.g. based on a variance of such predictions by sub-models 110a, 110b, 110c).

In at least some embodiments, loss determiner 130 determines a loss value of an objective function for a plurality of predictions 122a. For a given plurality of predictions 122a based on a corresponding plurality of synthetic spectral measurements 108a (from which machine learning model 110 generated predictions 122a), the loss value for the objective function may be based on predictions 122a and the synthetic soil properties 104a from which the corresponding plurality of synthetic spectral measurements 108a were generated. For instance, synthetic soil properties 104a, and/or information derived therefrom (e.g. soil properties of soil models generated therefrom by soil modeler 103), may be used as labels for the purpose determining a value of an objective function by loss determiner 130 in the course of training machine learning model 110.

Parameter trainer 138 trains parameters of model 110 based on the value of the objective function determined by loss determiner 130. Training parameters of model 110 may comprise determining updated parameters via an optimization algorithm, such as gradient descent. For instance, parameter trainer 138 may determine updated parameters based on a gradient of the objective function with respect to the parameters (sometimes denoted ∇μ/∇θ for an objective function μ and parameters θ).

System 100a may train parameters of model 110 on synthetic spectral measurements 108a, such as synthetic Raman spectral measurements, over a plurality of batches and/or epochs and may stop by applying a stopping criterion 132. Stopping criterion 132 may comprise completing a predetermined (e.g. user-provided) number of batches and/or epochs, achieving a target loss value (at loss determiner 130), achieving a target gradient (at parameter trainer 138), and/or any other suitable criterion. As one non-limiting example, in the example embodiment of FIG. 1A, stopping criterion 132 is based on a loss value determined by loss determiner 130.

When system 100a stops training model 110, the parameters of model 110 as trained by parameter trainer 138 may be output, stored, or otherwise provided as trained parameters 136a.

In some embodiments, system 100a trains only a portion of model 110. For example, system 100a may train a first portion of model 110 which receives spectral measurements as input but which does not receive certain other inputs of model 110. Model 110 may comprise a second portion which receives input based on the output of the first portion and one or more further inputs, such as location identifiers (e.g. location identifiers 109, described in greater detail with reference to FIG. 1B). For example, the first component of model 110 may comprise a convolutional neural network where convolutions (e.g. one-dimensional convolutions) are applied to input spectral measurements, such as synthetic spectral measurements 108a and ground-truth spectral measurements 108b (described in greater detail with reference to FIG. 1B). The output of the first component may be transformed to a prediction 122a by applying a prediction transformation, such as by transforming the output of the first component by a fully-connected layer. The first component network and prediction transformation may be trained by system 100a as described above.

Where there is a desire to train model 110 over further inputs which are not provided for synthetic spectral measurements 108b and/or which are not provided by system 100a, the output of the first component may be combined with such further inputs (e.g. location identifiers, terrain representations, and/or other covariate data) and passed through the second component of model 110 to generate prediction 122b, as described in greater detail with reference to FIG. 1B. Combination of inputs may comprise concatenation, a non-linear combination (e.g. via a neural network), and/or any other suitable combination. The second component may, for example, comprise a second neural network (e.g. comprising a fully connected layer) trained to generate prediction 122b as output. The prediction transformation for generating prediction 122a is not necessarily applied when training the second component so as to preserve representational richness.

In some embodiments, system 100a trains all of model 110. For such embodiments where model 110 receives input other than spectral measurements, synthetic spectral measurements 108a may be associated with further synthetic inputs, such as synthetic location identifiers and/or any other inputs expected by model 110. Further synthetic inputs may be predetermined, e.g. provided by a user, and/or imputed by system 100a. For example, system 100a may determine a synthetic location for a given spectral measurement 108a by generating a location based on an input spectral measurement via a machine learning model trained over ground-truth spectral measurements and associated location identifiers (such as ground-truth spectral measurements 108b and location identifiers 109, described in greater detail with reference to FIG. 1B, and/or based on other spectral measurements and location identifiers, such as those obtained from regional soil characteristics datasets). In some embodiments, a synthetic location for a given spectral measurement 108a is based on synthetic soil properties 104a for the given spectral measurement 108a. For example, system 100a may associate synthetic soil properties 104a with a location and/or a region based on similarity of synthetic soil properties 104a to soil properties associated with the location and/or region, e.g. by clustering locations and/or regions in a soil properties database based on soil properties, associating synthetic soil properties 104a for the given spectral measurement 108a with a cluster of locations and/or regions, and selecting a location from the cluster of locations and/or regions (e.g. by random sampling and/or any other suitable selection approach) to use as the synthetic location. A synthetic location identifier for the synthetic location (e.g. longitude and latitude coordinates) may be provided to model 110 as input.

In some embodiments, intermediate features are generated based on synthetic soil measurements 108a and/or other inputs to machine learning model 110 and may be provided as input to machine learning model 110. For example, intermediate features may be generated by applying spectral transformations, dimensionality transformations, and/or other transformations to synthetic soil measurements 108a. Such intermediate features may be provided to machine learning model 110 instead of, or in addition to, synthetic soil measurement 108a.

FIG. 1B schematically shows an example system 100b for training machine learning model 110 in a second training mode based on ground-truth soil samples. Systems 100a and 100b may be provided a system 100 and/or by different systems. In some embodiments, model 110 is initialized with trained parameters 136a by parameter loader 105 and system 100b further trains parameters of model 110 to generate trained parameters 136b (e.g. model 110 may be “pre-trained” by system 110 and “fine-tuned” by model 100b). In some embodiments, model 110 is trained by systems 100a and 100b in parallel and/or by interleaving, e.g. by alternating between training by system 100a and 100b.

System 100b trains machine learning model 110 over ground-truth spectral measurements 108b of the ground-truth soil samples. Ground truth spectral measurements 108b may comprise, for example, one or more of: Rayleigh, Raman, IR, and/or other spectral measurements. In some embodiments, ground truth spectral measurements 108b comprise Raman spectral measurements. Ground-truth spectral measurements 108b are associated with ground-truth training data comprising measured soil properties 104b of the ground-truth soil samples. Measured soil properties 104b may comprise, for example, measures of soil properties obtained via chemical analysis and/or any other suitable approach for characterizing soil properties. Measures of soil properties may comprise, for example, measures of soil carbon, such as measures of soil carbon based on whether the soil carbon is mineral-associated, permanent, stable, or otherwise durable. For instance, measured soil properties 104b may comprise a measure of mineral associated organic matter (MAOM) content in ground-truth soil samples. Measured soil properties 104b may additionally, or alternatively, comprise measures of other soil properties, such as non-sequestered soil carbon content (e.g. particulate organic matter, or POM, content); total soil carbon content; nitrogen, potassium, sulfur, hydrogen, nitrate, ammonia, phosphate, aluminum, iron, phosphorus, calcium, magnesium, and/or sodium content; phospholipid fatty acid content; pH; carbon-nitrogen ratio; Haney test inputs and outputs (which may include certain of the foregoing and/or other features such as water-soluble organic carbon and/or organic nitrogen); and/or other soil properties.

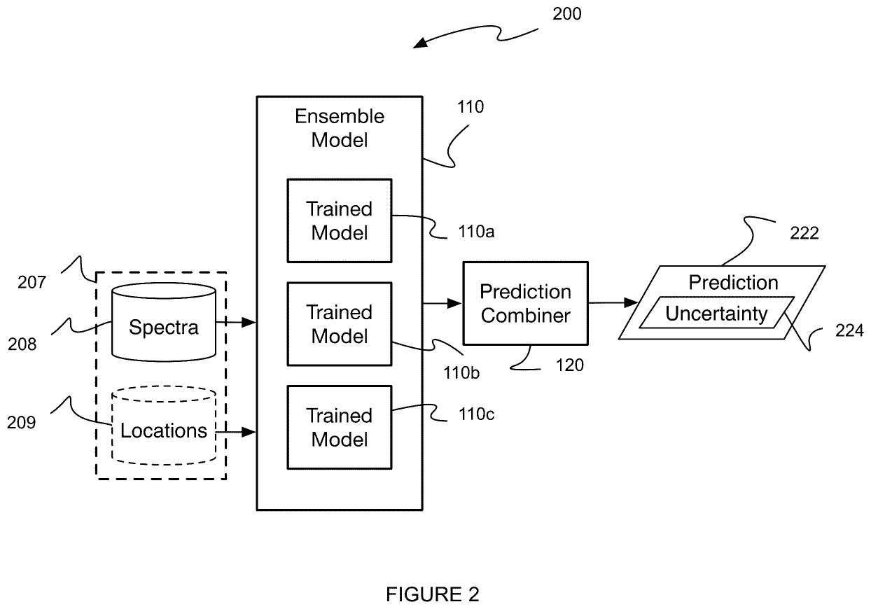

Spectral measurements 108b may be associated with other inputs for model 110, together forming inputs 107. In some embodiments, inputs 107 comprise location identifiers 109 associated with ground-truth spectral measurements 108b. Location identifiers 109 represent locations of ground-truth soil samples, such as a latitude and longitude of the location where a ground-truth soil sample was collected. In at least some embodiments, inputs 107 have the same form as inputs 207 (described elsewhere herein with respect to FIG. 2).

System 100b generates predictions 122b via machine learning model 110 based on inputs 107, including ground-truth spectral measurements 108b. Machine learning model 110 receives a ground-truth spectral measurement 108b and other inputs 107 (if any) and generates a prediction 122b of soil properties in the simulated soil sample associated with the received ground-truth spectral measurement 108b and other inputs 107 (if any). Prediction 122b may additionally, or alternatively, comprise predictions of other soil properties, such as nitrogen, potassium, sulfur, hydrogen, nitrate, ammonia, phosphate, aluminum, iron, phosphorus, calcium, magnesium, and/or sodium content; phospholipid fatty acid content; pH; carbon-nitrogen ratio; Haney test inputs and outputs (which may include certain of the foregoing and/or other features such as water-soluble organic carbon and/or organic nitrogen); and/or other soil properties.

In at least some embodiments, machine learning model 110 is configured to provide a measure of uncertainty 124b for prediction 122b, e.g. substantially as described elsewhere herein. For instance, machine learning model 110 may comprise a Bayesian ensemble neural network configured to provide a measure of uncertainty 124b for prediction 122b based on predictions by sub-models 110a, 110b, 110c (e.g. based on a variance of such predictions by sub-models 110a, 110b, 110c).

System 100b trains parameters of model 110 based on prediction 122b. In at least some embodiments. System 100b trains parameters of model 110 based on prediction 122b substantially as described above with reference to system 100a and prediction 122a of FIG. 1A. System 100b provides trained parameters 136b for model 110 as a result of said training.

In some embodiments, spectral measurements 108b comprise high-spatial-resolution spectral measurements and low-spatial-resolution spectral measurements. For example, low-spatial-resolution spectral measurements may comprise satellite images and/or aerial images of an area of interest. Such images may be associated with location identifiers on the basis of which system 100b may determine location identifiers for spectral measurements 108b (e.g. further based on a location of a spectral measurement within the image and a resolution of the image). As another example, high-resolution spectral measurements may comprise at-depth spectral measurement of soil, such as spectral measurements of soil samples obtained at a depth of about 0.01 m to 1 m, 0.1 m to 1 m, 0.2 m to 1 m, 0.3 m to 1 m, 0.4 m to 1 m, 0.5 m to 1 m, 0.25 m to 1 m, 0.25 m to 0.75 m, 0.25 m to 0.5 m, 0.5 m to 1 m, 0.1 m to 10 m, 0.1 m to 5 m, and/or 0.1 m to 2 m.

Spectral measurements may comprise Rayleigh, Raman, IR, and/or other spectral measurements. For example, in some embodiments the high-resolution spectral measurements comprise Raman spectral and/or IR spectral measurements of soil samples obtained proximally at depth (and may optionally include Rayleigh spectral measurements), and the low-resolution spectral measurements comprise Rayleigh measurements obtained aerially. For instance, a proximal sensor (e.g. a handheld sensor and/or a vehicle-mounted sensor) may measure spectra of soil at various locations throughout the area of interest. As another example, soil samples may be collected from the area of interest, returned to a lab, and their spectra may be measured there with laboratory spectrometers. (Other soil measurements, e.g. non-spectral measurements, may optionally be obtained as well.)

As noted above, in some embodiments, such as embodiments where inputs 107 provide more information than is provided during training by system 100a, model 110 may comprise first and second components. The first component may be trained by system 100a and the second component may be trained by system 100b. Such training by system 100b may comprise generating an intermediate representation by the first component, as trained by system 100a, combining inputs 107 not received by the first component with the intermediate representation (e.g. by concatenation), and transforming such combination to prediction 122b by the second component of model 110. The second component may, for example, comprise a second neural network (e.g. comprising a fully connected layer).

In some embodiments, training model 110 may comprise performing semi-supervised training. Such training may comprise providing unlabeled data, e.g. by providing at least some soil measurements 108a and/or 108b without providing and/or training over ground-truth training data such as measured soil properties 104b. Such training may comprise performing techniques such as self-training, co-training, and/or autoencoder-based pretraining to model 110 based on soil measurements 108a and/or 108b.

A machine learning model 110 may be trained by one or both of systems 100a and 100b. For example, a system may train machine learning model 110 based on synthetic spectral measurements without necessarily training machine learning model 110 based on ground-truth spectral measurements. As another example, a system may train machine learning model 110 based on ground-truth spectral measurements without necessarily training machine learning model 110 based on synthetic spectral measurements.

At least one of systems 100a and 100b train machine learning model 110 based on Raman spectral measurements. In some embodiments, machine learning model 110 is trained based on synthetic Raman spectral measurements and is not necessarily trained on ground-truth Raman spectral measurements. In some embodiments, machine learning model 110 is trained based on ground-truth Raman spectral measurements and is not necessarily trained on synthetic Raman spectral measurements. In some embodiments, machine learning model 110 is trained based on non-Raman spectral measurements by systems 100a and 100b, and is further trained on Raman spectral measurements by one of systems 100a and 100b (without necessarily being trained on Raman spectral measurements by the other of systems 100a and 100b).

### Systems for Predicting Soil Properties with a Model

FIG. 2 schematically shows an example system 200 for generating predictions of soil properties based on Raman spectral measurements with the example machine learning model 110 of FIGS. 1A and 1B.

System 200 obtains input data 207, e.g. from a user, from a data store, or via any suitable source. Input data 207 comprises an input Raman spectral measurement 208 of a soil sample. Input data 207 may optionally comprise other data, such as a location identifier 209 associated with input Raman spectral measurement 208, e.g. substantially as described elsewhere herein with reference to location identifier 109 and ground-truth spectral measurement 108b. Input data 207 may alternatively or additionally comprise representations of terrain characteristics, such as slopes/gradients, aspect, convergence index, hill shade, multi-resolution right top flatness, multi-resolution valley bottom flatness, standardized water level index, valley depth, elevation, different from the mean elevation, deviation from the mean elevation, relative terrain index, profile curvature, planform curvature, and/or other characteristics of terrain at the location of the soil sample.

System 200 generates, by machine learning model 110 and based on the trained parameters of machine learning model 110 (such as trained parameters 136a, 136b) a prediction 222 of soil properties for the soil sample associated with Raman spectral measurements 208. The trained parameters of machine learning model 110 were trained over synthetic soil properties training data and associated synthetic training spectral measurements (e.g. substantially as described above with respect to synthetic spectral measurements 108a and system 100a of FIG. 1A) and ground truth soil properties training data and associated ground truth training spectral measurements (e.g. substantially as described above with respect to ground-truth spectral measurements 108b and system 100b of FIG. 1B). Model 110 may comprise an ensemble model and system 200 may comprise a prediction combiner 120 for combining predictions of sub-models 110a, 110b, 110c to form prediction 222, e.g. substantially as described elsewhere herein.

In at least some embodiments, machine learning model 110 is configured to provide a measure of uncertainty 224 for prediction 222, e.g. substantially as described elsewhere herein. For instance, machine learning model 110 may comprise a Bayesian ensemble neural network configured to provide a measure of uncertainty 224 for prediction 222 based on predictions by sub-models 110a, 110b, 110c (e.g. based on a variance of such predictions by sub-models 110a, 110b, 110c).

### Methods for Training a Soil Properties Prediction Model

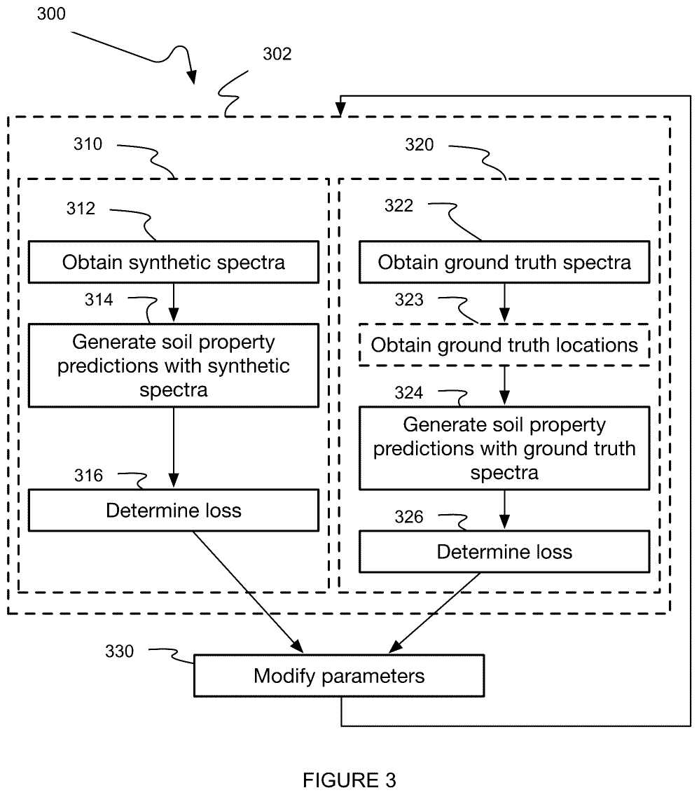

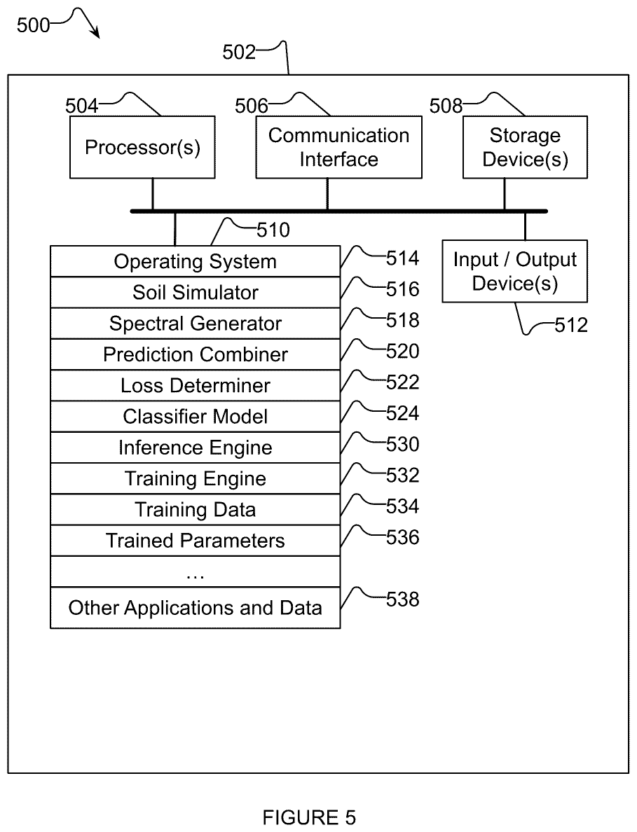

FIG. 3 is a flowchart of an example method 300 for training an example machine learning model (such as machine learning model 110) for predicting soil properties content, such as by systems 100a, 100b of FIGS. 1A and 1B. Method 300 is performed by a processor, e.g. as described with reference to system 500 of FIG. 5.

In some embodiments, method 300 comprises acts 302 for training the machine learning model over synthetic and/or ground-truth Raman spectral measurements. Acts 302 may comprise acts 310 for training the machine learning model over synthetic Raman spectral measurements and/or acts 320 for training the machine learning model over ground-truth Raman spectral measurements. Acts 310 and 320, where both are provided by method 300, may be performed in series (e.g. by performing acts 310 to completion and then performing acts 320 to completion), in parallel (e.g. by training the machine learning model on both synthetic and ground-truth spectral measurements in a given batch), by interleaving (e.g. by training via acts 310, followed by acts 320, followed by acts 310, followed by acts 320, and so on), and/or in any other suitable order. Such training, in the case of each of acts 310 and 320, may comprise modifying parameters as described with reference to act 330.

Turning to acts 310, at act 312, the processor obtains synthetic Raman spectral measurements for a plurality of simulated soil samples, e.g. as described with reference to synthetic spectral measurements 108a of FIG. 1A. Optionally, the processor may further obtain reflectance, IR, and/or other forms of spectra.

At act 314, the processor generates a plurality of predictions of soil properties content for the simulated soil samples obtained at act 312. The processor generates the plurality of predictions based on a machine learning model (referred to herein for disambiguation as a “prediction model”) based on synthetic Raman spectral measurements, and optionally other inputs (e.g. location identifiers, terrain representations, other spectral measurements, and/or other covariate data). Generating the plurality of predictions may comprise, for example, transforming the synthetic Raman spectral measurements based on trained parameters of the prediction model to form a prediction of soil properties for the simulated soil sample, e.g. as described with reference to prediction 122a of FIG. 1A. In some embodiments act 314 comprises generating an intermediate prediction suitable for use by acts 324 and/or 404 and transforming the intermediate prediction to a prediction, e.g. as described elsewhere herein with reference to FIGS. 1A and 1B. The prediction model may comprise, for example, a regressor and/or classifier machine learning model.

At act 316, the processor determines a value of an objective function based on the plurality of predictions of soil properties and synthetic soil properties training data associated with the synthetic Raman spectral measurements. For example, the synthetic soil properties training data may comprise a measure of soil properties (such as sequestered soil carbon and/or other characteristics), e.g. as described above with reference to synthetic soil properties 104a. The processor may determine the value of the objective function as described elsewhere herein with reference to loss determiner 130 of FIG. 1A.

In some embodiments, act 302 of method 300 comprises acts 320 for training parameters of the machine learning model based on ground-truth Raman spectral measurements. For example, at act 322 the processor obtains ground-truth Raman spectral measurements for a plurality of ground-truth soil samples, e.g. as described with reference to ground-truth spectral measurements 108b of FIG. 1B.

Optionally, at act 323, the processor obtains location identifiers associated with the ground-truth spectral measurements obtained at act 322. For example, the processor may obtain location identifiers 109 associated with ground-truth spectral measurements 108b, e.g. as described with reference to FIG. 1B.

At act 324, the processor generates a plurality of predictions of soil properties for the soil samples based on the ground truth training Raman spectral measurements obtained at act 322. The processor generates the plurality of predictions based on a prediction model based on the ground-truth Raman spectral measurements, such as by transforming the ground-truth Raman spectral measurements based on trained parameters of the prediction model to form a prediction of soil properties for the simulated soil sample, e.g. as described with reference to prediction 122b of FIG. 1B.

In some embodiments, act 324 comprises providing the prediction model with additional inputs, such as location identifiers associated with the ground-truth spectral measurements, IR spectral measurements, Rayleigh spectral measurements, and/or other inputs. Other inputs may alternatively or additionally comprise representations of terrain characteristics, such as slopes/gradients, aspect, convergence index, hill shade, multi-resolution right top flatness, multi-resolution valley bottom flatness, standardized water level index, valley depth, elevation, different from the mean elevation, deviation from the mean elevation, relative terrain index, profile curvature, planform curvature, and/or other characteristics of terrain at the location of the soil sample. For example, act 324 may comprise generating an intermediate representation based on a portion of the input data (e.g. as described elsewhere herein with reference to act 314), combining the intermediate representation with additional inputs, and generating a prediction based on the combined inputs (e.g. as described elsewhere herein with reference to FIGS. 1A and 1B.

At act 326, the processor determines a value of an objective function based on the plurality of predictions of soil properties and ground-truth soil properties training data associated with the ground-truth Raman spectral measurements. For example, the ground-truth soil properties training data may comprise a measure of soil properties (such as sequestered soil carbon and/or other characteristics), e.g. as described above with reference to ground-truth soil properties 104b. The processor may determine the value of the objective function as described elsewhere herein with reference to loss determiner 130 of FIG. 1B.

At act 330, the processor modifies parameters of the prediction model based on one or more values of the objective functions obtained at acts 316 and/or 326. Modifying the parameters of the prediction model may comprise, for example, determining updated parameters via an optimization algorithm, such as gradient descent, e.g. as described with reference to parameter trainer 138 of FIGS. 1A and 1B.

### Methods for Predicting Soil Properties with a Model

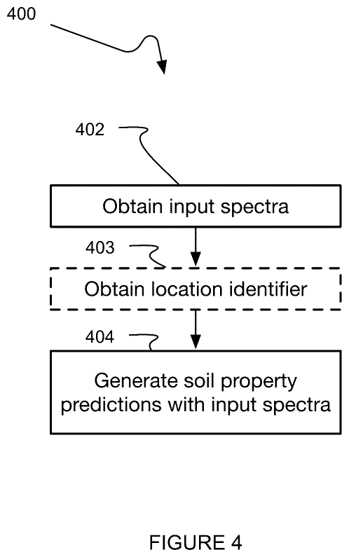

FIG. 4 is a flowchart of an example method 400 for generating predictions of soil properties with an example machine learning model (such as machine learning model 110) for predicting soil properties based on Raman spectral measurements, such as by the system 200 of FIG. 2. Method 400 is performed by a processor, e.g. as described with reference to system 500 of FIG. 5.

At act 402, the processor obtains input Raman spectral measurements for soil at a location, e.g. as described with reference to input Raman spectral measurements 208 of FIG. 2.

Optionally, at act 403, the processor obtains a location identifier associated with the input Raman spectral measurement obtained at act 402. For example, the processor may obtain a location identifier 209 associated with input Raman spectral measurement 208, e.g. as described with reference to FIG. 2.

At act 404, the processor generates prediction of soil properties for the soil associated with the input Raman spectral measurement obtained at act 402. The processor generates the prediction by a prediction model based on the input Raman spectral measurement. The prediction model has trained parameters trained over synthetic soil properties training data (such as synthetic soil properties 104a) and associated synthetic training Raman spectral measurements (such as synthetic spectral measurements 108a) and, optionally, based on ground-truth soil properties training data (such as ground-truth soil properties 104b) and associated ground-truth training Raman spectral measurements (such as ground-truth spectral measurements 108b). For instance, the trained parameters of the prediction model may be trained as described according to method 300. Training the trained parameters of the prediction model is not necessarily included within the scope of method 400, although such acts may optionally be included.

Act 404 optionally comprises providing the prediction model with additional inputs, such as location identifiers, terrain representations, and/or other covariate data as described in greater detail elsewhere herein. Generating a prediction may comprise, for example, transforming the input Raman spectral measurements based on trained parameters of the prediction model to form a prediction of soil properties for the soil associated with the input Raman spectral measurement, e.g. as described with reference to prediction 222 of FIG. 2. In some embodiments act 404 comprises generating an intermediate prediction by a first component of the prediction model based on the input Raman spectral measurement (and, optionally, other inputs) and generating a prediction based on the intermediate representation and further inputs by a second component of the prediction model, e.g. as described in greater detail elsewhere herein.

In at least some embodiments, act 404 comprises generating a measure of uncertainty for the prediction. For instance, the prediction model may comprise a Bayesian ensemble neural network configured to provide a measure of uncertainty for predictions based on predictions by sub-models of the ensemble model (such as sub-models 110a, 110b, 110c of model 110), e.g. as described in greater detail elsewhere herein.

### Computer System

FIG. 5 illustrates a first exemplary operating environment 500 that includes at least one computing system 502 for performing methods described herein. System 502 may be any suitable type of electronic device, such as, without limitation, a mobile device, a personal digital assistant, a mobile computing device, a smart phone, a cellular telephone, a handheld computer, a server, a server array or server farm, a web server, a network server, a blade server, an Internet server, a work station, a mini-computer, a mainframe computer, a supercomputer, a network appliance, a web appliance, a distributed computing system, multiprocessor systems, or combination thereof. System 502 may be configured in a network environment, a distributed environment, a multi-processor environment, and/or a stand-alone computing device having access to remote or local storage devices.

A computing system 502 may include one or more processors 504, a communication interface 506, one or more storage devices 508, one or more input and output devices 512, and a memory 510. A processor 504 may be any commercially available or customized processor and may include dual microprocessors and multi-processor architectures. A processor 504 may comprise a CPU, a GPU, and/or any other suitable processor. Terms such as “a processor” and “the processor” include a plurality of processors. The communication interface 506 facilitates wired or wireless communications between the computing system 502 and other devices. A storage device 508 may be a computer-readable medium that does not contain propagating signals, such as modulated data signals transmitted through a carrier wave. Examples of a storage device 508 include without limitation RAM, ROM, EEPROM, flash memory or other memory technology, CD-ROM, digital versatile disks (DVD), or other optical storage, magnetic cassettes, magnetic tape, magnetic disk storage. In at least some embodiments such embodiments of storage device 508 do not contain propagating signals, such as modulated data signals transmitted through a carrier wave. There may be multiple storage devices 508 in the computing system 502. The input/output devices 512 may include a keyboard, mouse, pen, voice input device, touch input device, display, speakers, printers, etc., and any combination thereof.

The memory 510 may be any non-transitory computer-readable storage media that may store executable procedures, applications, and data. The computer-readable storage media does not pertain to propagated signals, such as modulated data signals transmitted through a carrier wave. It may be any type of non-transitory memory device (e.g., random access memory, read-only memory, etc.), magnetic storage, volatile storage, non-volatile storage, optical storage, DVD, CD, floppy disk drive, etc. that does not pertain to propagated signals, such as modulated data signals transmitted through a carrier wave. The memory 510 may also include one or more external storage devices or remotely located storage devices that do not pertain to propagated signals, such as modulated data signals transmitted through a carrier wave.

The memory 510 may contain instructions, components, and data. A component is a software program that performs a specific function and is otherwise known as a module, program, engine, and/or application. The memory 510 may include an operating system 514, a soil simulator 516 (e.g. providing a soil simulator 102), a spectral generator 518 (e.g. providing a spectral generator 106), a prediction combiner 520 (e.g. providing a prediction combiner 120), a loss determiner 522 (e.g. providing a loss determiner 130), a classifier model (e.g. providing an ensemble model 110), an inference engine 530 (e.g. for performing inference via method 400), a training engine 532 (e.g. for performing training via method 300), training data 532 (e.g. comprising synthetic and/or ground-truth spectral measurements, soil properties, and/or other data as described in greater detail elsewhere herein), trained parameters 536 (e.g. comprising parameters 136a and/or 136b), and other applications and data 538.

Depending on the embodiment, some such elements may be wholly or partially omitted. For example, an embodiment configured for inference (e.g. via method 400 and/or system 200) but not necessarily for training might omit training data 534, soil simulator 516, spectral generator 518, loss determiner 522, and/or training engine 532. As another example, an embodiment configured for training (e.g. via method 300 and/or systems 100a, 100b) but not necessarily for inference might omit inference engine 530. In some embodiments configured for training, memory 510 may omit trained parameters 536 prior to starting training and may generate such trained parameters 536 over the course of training. Some or all components may be obtained via an input device 512, communication interface 506, and/or storage device 508.

### Conclusion

While a number of exemplary aspects and embodiments have been discussed above, those of skill in the art will recognize certain modifications, permutations, additions, and sub-combinations thereof. It is therefore intended that the following appended claims and claims hereafter introduced are interpreted to include all such modifications, permutations, additions and sub-combinations as are within their true spirit and scope.

What is claimed is:

1. A method for predicting soil properties, the method performed by a processor and comprising:
obtaining an input Raman spectral measurement of soil;
generating a first prediction of a soil property for the soil by a machine learning model configured to generate predictions of the soil property based on input Raman spectral measurement and having parameters trained over soil property training data and associated soil property training Raman spectral measurements.

2. The method according to claim 1 wherein the Raman training data and associated Raman training spectral measurements comprise:
synthetic sequestered soil property training data and associated synthetic training Raman spectral measurements; and
ground truth sequestered soil property training data and associated ground truth training Raman spectral measurements.

3. The method according to claim 2 wherein the synthetic soil property training data comprises a measure of the soil property in a plurality of simulated soil samples, and the ground truth soil property training data comprises a measure of the soil property in a plurality of ground-truth soil samples.

4. The method according to claim 2 wherein the synthetic training Raman spectral measurements comprises a simulated Raman spectral measurement generated by a physical soil simulation.

5. The method according to claim 4 wherein at least one simulated Raman spectral measurement for a physical soil simulation comprising first and second soil components is based on a sum of a first simulated Raman spectral measurement for the first soil component and a second simulated Raman spectral measurement for the second soil component.

6. The method according to claim 2 wherein the simulated Raman spectral measurement is based on a simulation of at least one of: a vibrational frequency and a vibrational intensity of molecules represented in the physical soil simulation.

7. The method according to claim 2 wherein the synthetic training Raman spectral measurement comprises a predicted Raman spectral measurement generated by a generative machine learning model trained over soil properties and configured to predict Raman spectral measurements based on soil properties.

8. The method according to claim 2 wherein:
the ground truth soil property training data corresponds to a plurality of ground truth training locations in an area of interest;
the machine learning model is configured to generate predictions of the soil property based on the input Raman spectral measurement and a location identifier associated with the input Raman spectral measurement, the machine learning model having parameters trained over a plurality of ground-truth location identifiers associated with the ground truth training Raman spectral measurements; and
generating the first prediction of the soil property for the soil comprises generating the first prediction of the soil property for the location based on a location identifier for the soil associated with the input Raman spectral measurement.

9. The method according to claim 8 wherein:
the first portion of the machine learning model has parameters trained over synthetic soil property training data and associated synthetic training Raman spectral measurements; and
the second portion of the machine learning model has parameters trained over synthetic soil property training data and associated synthetic training Raman spectral measurements and ground-truth soil property training data and associated ground-truth training Raman spectral measurements.

10. The method according to claim 1 wherein the input Raman spectral measurement comprises a first Raman spectral signature corresponding to mineral-associated organic material and a second Raman spectral signature corresponding to particulate organic material, the method further comprising distinguishing at least a portion of the first spectral signature from the second spectral signature.

11. The method according to claim 1 wherein the first prediction of the soil property comprises a predicted measure at least one of: soil carbon content, nitrogen content, potassium content, sulfur content, hydrogen content, nitrate content, ammonia content, phosphate content, aluminum content, iron content, phosphorus content, calcium content, magnesium content, sodium content, phospholipid fatty acid content, pH, carbon-nitrogen ratio, water content, water-soluble organic carbon content, and water-soluble organic nitrogen content.

12. The method according to claim 11 wherein the first prediction of the soil property comprises a predicted measure of sequestered soil carbon content for the soil.

13. The method according to claim 11 wherein the predicted measure of sequestered soil carbon content comprises a measure of mineral associated organic matter content.

14. The method according to claim 11 further comprising generating a second prediction of non-sequestered soil carbon content for the soil.

15. The method according to claim 14 further comprising generating a third prediction of total soil carbon content for the soil, the third prediction of total soil carbon content being based on a sum of soil carbon content of the first and second predictions.

16. The method according to claim 1 wherein the input Raman spectral measurement comprises a high-spatial-resolution spectral measurement and a low-spatial-resolution spectral measurement, at least one of the high-spatial-resolution spectral measurement and low-spatial-resolution spectral measurement comprising a Raman spectral measurement.

17. The method according to claim 16 wherein the low-spatial-resolution spectral measurement comprises at least one of: satellite images and aerial images of an area of interest comprising a location of the soil.

18. The method according to claim 16 wherein the high-resolution spectral measurement comprises an at-depth Raman spectral measurement of the soil.

19. The method according to claim 18 wherein the at-depth Raman spectral measurement comprises a spectral measurement of a soil sample obtained at a depth of at least one of: about 0.01 m to 1 m, 0.1 m to 1 m, 0.25 m to 1 m, and 0.1 m to 10 m.

20. The method according to claim 1 wherein the first prediction comprises a measure of uncertainty.
